(19) United States
(12) Patent Application Publication
(10) Pub. No.: US 2024/0383899 A1
(43) Pub. Date: Nov. 21, 2024

(54) MODIFIED CARBAPENEM ANTIBIOTICS WITH IMPROVED ACTIVITY AGAINST KPC CARBAPENEMASE-PRODUCING ENTEROBACTERALES

(71) Applicant: Southern Methodist University, Dallas, TX (US)

(72) Inventors: John D. Buynak, Dallas, TX (US); Taariq Hassan, Murphy, TX (US); Pojun Quan, Commerce, TX (US)

(21) Appl. No.: 18/659,427

(22) Filed: May 9, 2024

Related U.S. Application Data
(60) Provisional application No. 63/502,211, filed on May 15, 2023.

(51) Int. Cl. C07D 477/20; A61K 31/407; A61K 45/06; A61P 31/04

(52) U.S. Cl. CPC C07D 477/20; A61K 31/407; A61K 45/06; A61P 31/04

(57) ABSTRACT

Provided herein are the design, preparation, evaluation, and use of structurally modified carbapenem antibiotics with improved activity, relative to current commercially available carbapenem antibiotics, against infections involving multidrug-resistant, carbapenemase-producing Enterobacterales strains. In particular, the new carbapenem antibiotics are demonstrated to produce improved activity, relative to current commercial carbapenem antibiotics, against bacterial strains producing a KPC carbapenemase. This invention includes their use as standalone antibiotics, as well as synergistic mixtures of the structurally modified carbapenems with carbapenemase inhibitors as well as synergistic mixtures of the newly modified carbapenems with other carbapenem antibiotics.

### CROSS-REFERENCE TO RELATED APPLICATIONS

This application claims priority to U.S. Provisional Application Ser. No. 63/502,211, filed May 15, 2023, the entire contents of which are incorporated herein by reference.

### STATEMENT OF FEDERALLY FUNDED RESEARCH

This invention was made with government support under AI142699 and AI155723 awarded by the National Institutes of Health. The government has certain rights in the invention.

### TECHNICAL FIELD OF THE INVENTION

The present invention relates in general to the field of antibiotics, and more particularly, to modified carbapenem antibiotics with improved activity against KPC carbapenemase-producing Enterobacterales.

### INCORPORATION-BY-REFERENCE OF MATERIALS FILED ON COMPACT DISC

Not applicable.

### BACKGROUND OF THE INVENTION

Without limiting the scope of the invention, its background is described in connection with Carbapenem Resistant Enterobacterales (CRE).

The 21st century has seen a rise in the incidence of Carbapenem Resistant Enterobacterales (CRE) leading to severe infections, including septicemia, complex urinary tract infections, and intra-abdominal infections. Microbial resistance to the beta-lactam antibiotics is most frequently mediated by the production of one or more beta-lactamases, enzymes that hydrolytically destroy beta-lactam antibiotics. Beta-lactamases that can hydrolyze carbapenem antibiotics are referred to as carbapenemases. The carbapenemase most frequently encountered among resistant strains of Enterobacterales is the Klebsiella pneumoniae (KPC) carbapenemase.¹ The KPC carbapenemase has an extremely broad spectrum of substrates, and is capable of hydrolyzing all current commercial beta-lactam antibiotics.

What is needed are novel compounds, methods, and combinations thereof, for the treatment of bacteria that hydrolyze carbapenem antibiotics.

### SUMMARY OF THE INVENTION

As embodied and broadly described herein, an aspect of the present disclosure relates to a compound of Formula I, or a pharmaceutically acceptable salt or ester thereof:

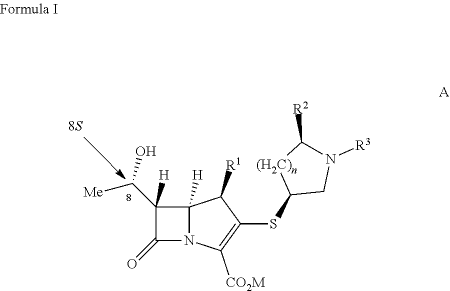

R¹=H or CH₃; n=0 or 1; R² is H, CH₂NHSO₂NH₂, or CONR^{a}R^{b}, where R^{a} and R^{b} may be independently equal to H, Me, Aryl, or substituted Aryl; and R³ is H, dihydrothiazole, C1 to C6 Alkyl, or R³=COR^{c}, where R^{c}=C1 to 6 alkyl, aryl, substituted aryl, or R^{c}=NHR^{d}, where R^{d}=H, aryl or substituted aryl. In one aspect, the compound is:

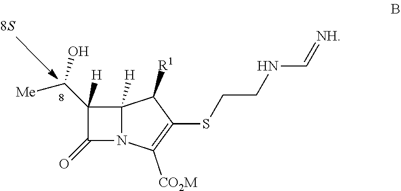

In another aspect, the —CO₂M, which is attached to the carbapenem nucleus at position 3, wherein M is a carboxylic acid group (M represents H), a carboxylate anion (M represents a negative charge), a pharmaceutically acceptable ester (M represents an ester forming group) or a carboxylic acid protected by a protecting group (M represents a carboxyl protecting group). In another aspect, the pharmaceutically acceptable salts form —COOM, where M is a negative charge, which is balanced by a counterion, an alkali metal cation, sodium, potassium calcium, magnesium, zinc, ammonium, or alkylammonium cations such as tetramethylammonium, tetrabutylammonium, choline, triethylhydroammonium, meglumine, or triethanolhydroammonium. In another aspect, the pharmaceutically acceptable salts comprise acid addition salts. In another aspect, the pharmaceutically acceptable salts comprise inorganic or organic acids. In another aspect, the pharmaceutically acceptable salts comprise acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, or undecanoate. In another aspect, the pharmaceutically acceptable ester is a hydrolyzable ester. In another aspect, the hydrolyzable esters is biologically hydrolyzable, is suitable for oral administration, has good absorption through the stomach or intestinal mucosa, or resistance to gastric acid degradation. In another aspect, the hydrolyzable esters M is selected from at least one of: an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl. In another aspect, the compound is:

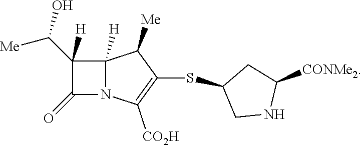

In another aspect, the compound is:

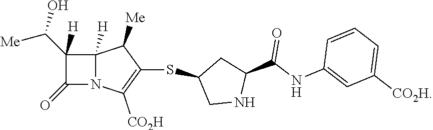 [SMILES: C[C@H](O)[C@H]1C(=O)N2C(C(=O)O)=C(S[C@@H]3CN[C@H](C(=O)Nc4cccc(C(=O)O)c4)C3)[C@H](C)[C@H]12]

In another aspect, the compound is:

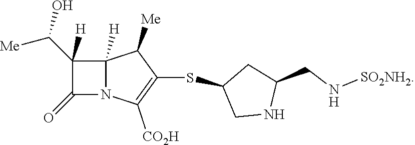 [SMILES: C[C@H](O)[C@H]1C(=O)N2C(C(=O)O)=C(S[C@@H]3CN[C@H](CNS(N)(=O)=O)C3)[C@H](C)[C@H]12]

In another aspect, the compound is:

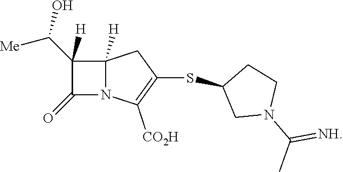 [SMILES: CC(=N)N1CC[C@H](SC2=C(C(=O)O)N3C(=O)[C@H]([C@H](C)O)[C@H]3C2)C1]

As embodied and broadly described herein, an aspect of the present disclosure relates to a method of making the compound of Formula I, comprising:

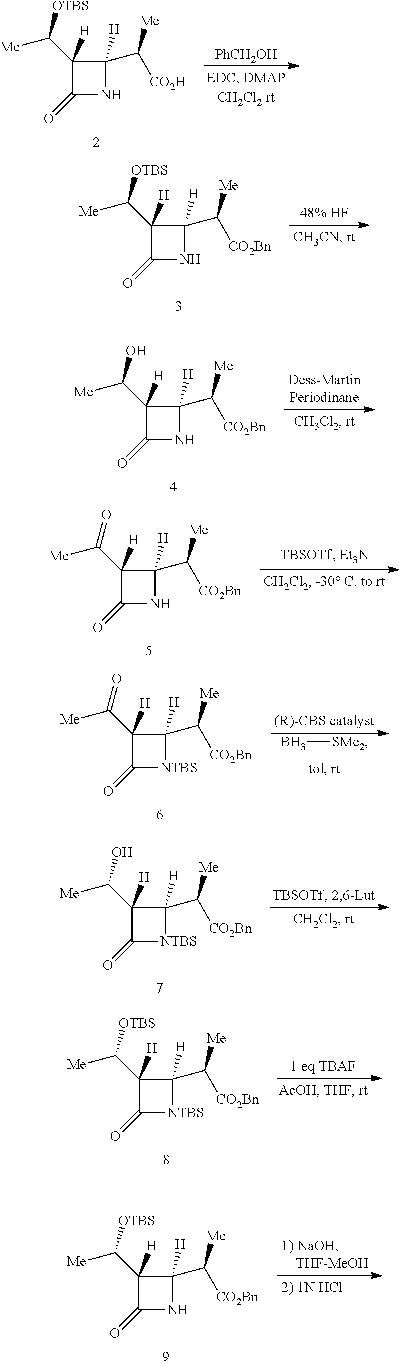

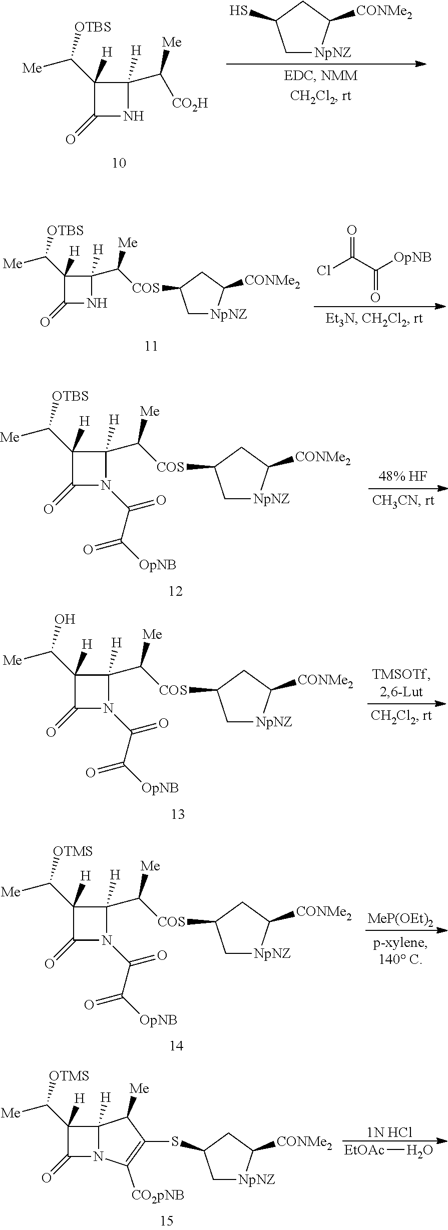

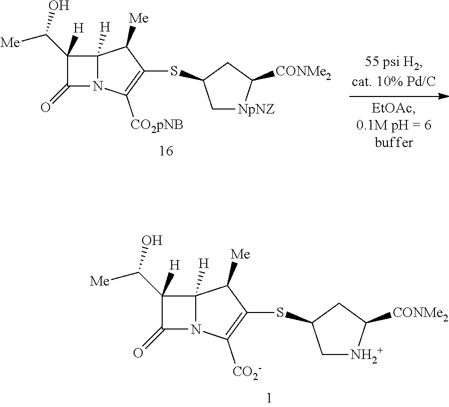

As embodied and broadly described herein, an aspect of the present disclosure relates to a composition comprising: a compound of Formula I, or a pharmaceutically acceptable salt or ester thereof:

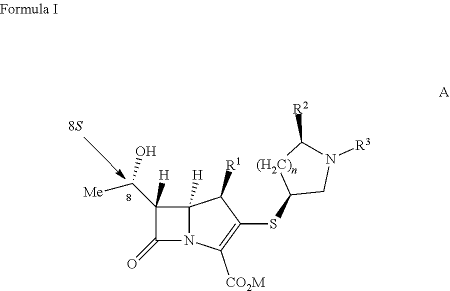

R¹=H or CH₃; n=0 or 1; R² is H, CH₂NHSO₂NH₂, or CONR^{a}R^{b}, where R^{a} and R^{b} may be independently equal to H, Me, Aryl, or substituted Aryl; and R³ is H, dihydrothiazole, C1 to C6 Alkyl, or R³=COR^{c}, where R^{c}=C1 to 6 alkyl, aryl, substituted aryl, or R^{c}=NHR^{d}, where R^{d}=H, aryl or substituted aryl; and a pharmaceutically acceptable carrier. In one aspect, the compound is:

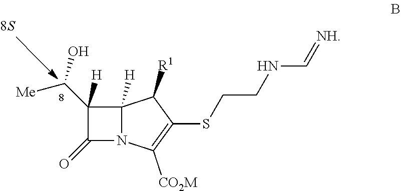

In another aspect, the —CO₂M, which is attached to the carbapenem nucleus at position 3, wherein M is a carboxylic acid group (M represents H), a carboxylate anion (M represents a negative charge), a pharmaceutically acceptable ester (M represents an ester forming group) or a carboxylic acid protected by a protecting group (M represents a carboxyl protecting group). In another aspect, the pharmaceutically acceptable salts form —COOM, where M is a negative charge, which is balanced by a counterion, an alkali metal cation, sodium, potassium calcium, magnesium, zinc, ammonium, or alkylammonium cations such as tetramethylammonium, tetrabutylammonium, choline, triethylhydroammonium, meglumine, or triethanolhydroammonium. In another aspect, the pharmaceutically acceptable salts comprise acid addition salts. In another aspect, the pharmaceutically acceptable salts comprise inorganic or organic acids. In another aspect, the pharmaceutically acceptable salts comprise acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, or undecanoate. In another aspect, the pharmaceutically acceptable ester is a hydrolyzable ester. In another aspect, the hydrolyzable esters is biologically hydrolyzable, is suitable for oral administration, has good absorption through the stomach or intestinal mucosa, or resistance to gastric acid degradation. In another aspect, the hydrolyzable esters M is selected from at least one of: an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyI, alkenylthioalkyl, arylthioalkyl or alkylthioaryI group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl. In another aspect, the compound is:

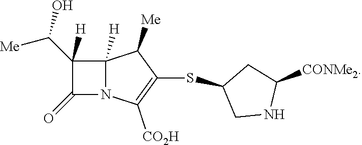

In another aspect, the compound is:

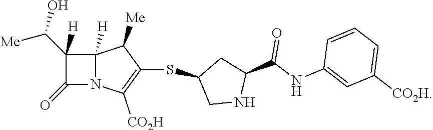 [SMILES: C[C@H](O)[C@H]1C(=O)N2C(C(=O)O)=C(S[C@@H]3CN[C@H](C(=O)Nc4cccc(C(=O)O)c4)C3)[C@H](C)[C@H]12]

In another aspect, the compound is:

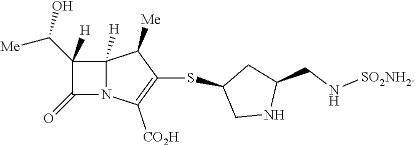 [SMILES: C[C@H](O)[C@H]1C(=O)N2C(C(=O)O)=C(S[C@@H]3CN[C@H](CNS(N)(=O)=O)C3)[C@H](C)[C@H]12]

In another aspect, the compound is:

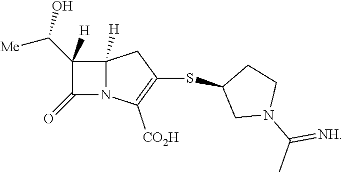 [SMILES: CC(=N)N1CC[C@H](SC2=C(C(=O)O)N3C(=O)[C@H]([C@H](C)O)[C@H]3C2)C1]

In another aspect, the composition further comprises an amount of a structurally modified carbapenem with carbapenemase inhibitors and synergistic mixtures of the compound of Formula I with other carbapenem antibiotics.

As embodied and broadly described herein, an aspect of the present disclosure relates to a method of treating a bacterial infection comprising: contacting a bacteria with a compound of Formula I, or a pharmaceutically acceptable salt or ester thereof:

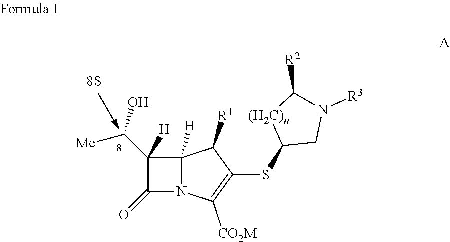

R¹=H or CH₃; n=0 or 1; R² is H, CH₂NHSO₂NH₂, or CONR^{a}R^{b}, where R^{a} and R^{b} may be independently equal to H, Me, Aryl, or substituted Aryl; and R³ is H, dihydrothiazole, C1 to C6 Alkyl, or R³=COR^{c}, where R^{c}=C1 to 6 alkyl, aryl, substituted aryl, or R^{c}=NHR^{d}, where R^{d}=H, aryl or substituted aryl; and a pharmaceutically acceptable carrier. In one aspect, the compound is:

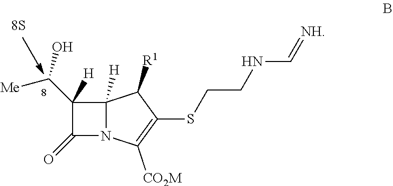

In another aspect, the —CO₂M, which is attached to the carbapenem nucleus at position 3, wherein M is a carboxylic acid group (M represents H), a carboxylate anion (M represents a negative charge), a pharmaceutically acceptable ester (M represents an ester forming group) or a carboxylic acid protected by a protecting group (M represents a carboxyl protecting group). In another aspect, the pharmaceutically acceptable salts form —COOM, where M is a negative charge, which is balanced by a counterion, an alkali metal cation, sodium, potassium calcium, magnesium, zinc, ammonium, or alkylammonium cations such as tetramethylammonium, tetrabutylammonium, choline, triethylhydroammonium, meglumine, or triethanolhydroammonium. In another aspect, the pharmaceutically acceptable salts comprise acid addition salts. In another aspect, the pharmaceutically acceptable salts comprise inorganic or organic acids. In another aspect, the pharmaceutically acceptable salts comprise acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, or undecanoate. In another aspect, the pharmaceutically acceptable ester is a hydrolyzable ester. In another aspect, the hydrolyzable esters is biologically hydrolyzable, is suitable for oral administration, has good absorption through the stomach or intestinal mucosa, or resistance to gastric acid degradation. In another aspect, the hydrolyzable esters M is selected from at least one of: an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl. In another aspect, the compound is:

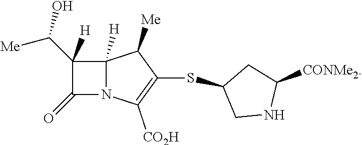

In another aspect, the compound is:

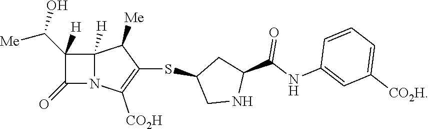 [SMILES: C[C@H](O)[C@H]1C(=O)N2C(C(=O)O)=C(S[C@@H]3CN[C@H](C(=O)Nc4cccc(C(=O)O)c4)C3)[C@H](C)[C@H]12]

In another aspect, the compound is:

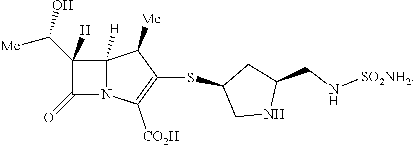 [SMILES: C[C@H](O)[C@H]1C(=O)N2C(C(=O)O)=C(S[C@@H]3CN[C@H](CNS(N)(=O)=O)C3)[C@H](C)[C@H]12]

In another aspect, the compound is:

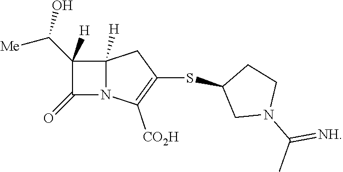 [SMILES: CC(=N)N1CC[C@H](SC2=C(C(=O)O)N3C(=O)[C@H]([C@H](C)O)[C@H]3C2)C1]

In another aspect, the method further comprises an amount of a structurally modified carbapenem with carbapenemase inhibitors and synergistic mixtures of the compound of Formula I with other carbapenem antibiotics.

### BRIEF DESCRIPTION OF THE DRAWINGS

For a more complete understanding of the features and advantages of the present invention, reference is now made to the detailed description of the invention along with the accompanying figures and in which:

FIG. 1 shows the general structures of atypical carbapenem antibiotic A.

FIG. 2 shows the general structures of atypical carbapenem antibiotic B.

FIG. 3 shows a synthetic procedure leading to carbapenem antibiotic 1 of general structure A.

FIG. 4 shows general structures of the present invention.

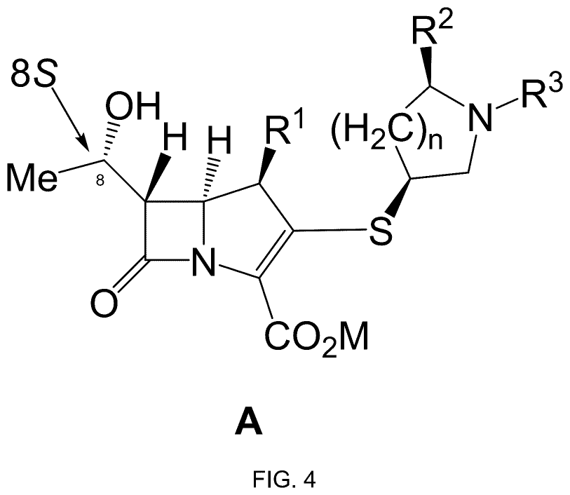

FIG. 5 shows a specific structure.

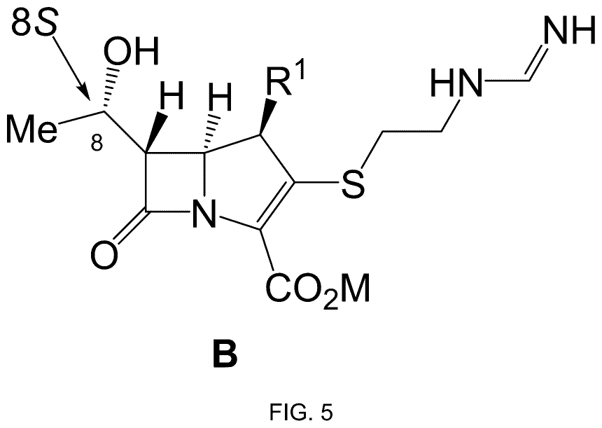

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Carbapenem-resistant Enterobacterales commonly produces a KPC carbapenemase, which is capable of hydrolyzing all commercial carbapenem antibiotics. A carbapenem antibiotic that is resistant to KPC-catalyzed hydrolysis would, therefore, be extremely useful in treatment of infections involving this pathogen. The currently described carbapenem antibiotics are not only resistant to hydrolysis, but will also inhibit the KPC carbapenemase, thus providing an opportunity to couple these new carbapenems with other b-lactam antibiotics, including, but not limited to, current commercial carbapenems, to attain enhanced potency in treatment of resistant Enterobacterales.

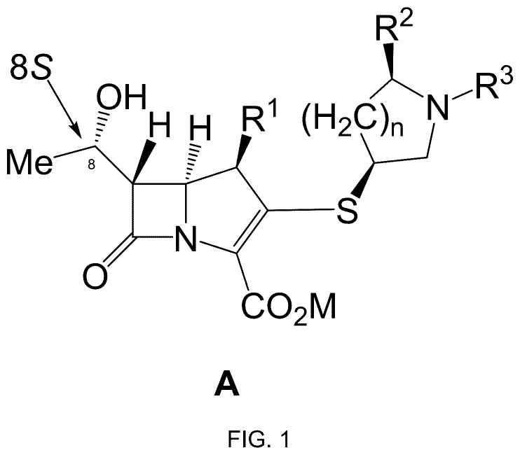

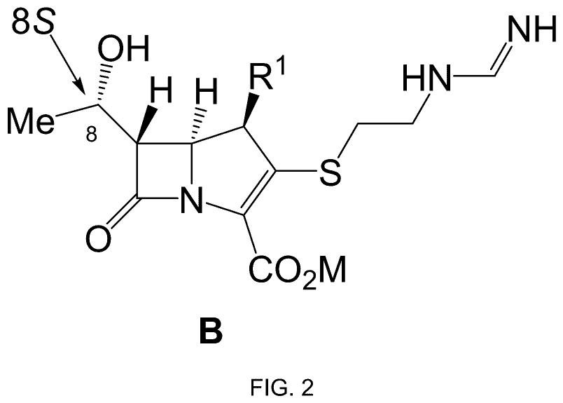

The General Structure of these carbapenems is shown in FIGS. 1 and 2.

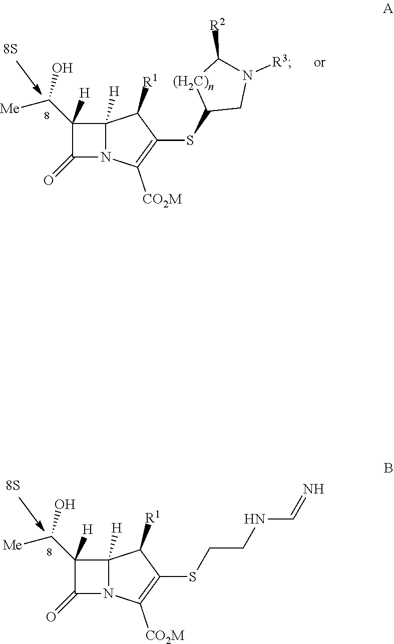

Wherein:
Where R¹=H or CH₃;
Where n=0 or 1;
Where R² may be H, CH₂NHSO₂NH₂, or CONR^{a}R^{b}, where R^{a} and R^{b} may be independently equal to H, Me, Aryl, or substituted Aryl;
Where R³ may be H, dihydrothiazole, C1 to C6 Alkyl, or R³=COR^{c}, where R^{c}=C1 to C6alkyl, aryl, substituted aryl, or R^{c}=NHR^{d}, where R^{d}=H, aryl or substituted aryl.

With respect to —CO₂M, which is attached to the carbapenem nucleus at position 3, this represents a carboxylic acid group (M represents H), a carboxylate anion (M represents a negative charge), a pharmaceutically acceptable ester (M represents an ester forming group) or a carboxylic acid protected by a protecting group (M represents a carboxyl protecting group).

The pharmaceutically acceptable salts referred to above may take the form —COOM, where M is a negative charge, which is balanced by a counterion, e.g., an alkali metal cation such as sodium or potassium. Other pharmaceutically acceptable counterions may be calcium, magnesium, zinc, ammonium, or alkylammonium cations such as tetramethylammonium, tetrabutylammonium, choline, triethylhydroammonium, meglumine, triethanolhydroammonium, etc.

The pharmaceutically acceptable salts referred to above also include acid addition salts. Thus, the Formula I compounds can be used in the form of salts derived from inorganic or organic acids. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

The pharmaceutically acceptable esters are such as would be readily apparent to a medicinal chemist, and include, for example, those described in detail in U.S. Pat. No. 4,309,438. Included within such pharmaceutically acceptable esters are those which are hydrolyzed under physiological conditions, such as pivaloyloxymethyl, acetoxymethyl, phthalidyl, indanyl and methoxymethyl, and others described in detail in U.S. Pat. No. 4,479,947. These are also referred to as “biolabile esters”.

Biolabile esters are biologically hydrolizable, and may be suitable for oral administration, due to good absorption through the stomach or intenstinal mucosa, resistance to gastric acid degradation and other factors. Examples of biolabile esters include compounds in which M represents an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl.

A dosage unit for use of the carbapenem antibiotic that is resistant to KPC-catalyzed hydrolysis of the present invention, may be a single compound or mixtures thereof with other compounds. The compound may be mixed together, form ionic or even covalent bonds. The carbapenem antibiotic that is resistant to KPC-catalyzed hydrolysis of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the carbapenem antibiotic that is resistant to KPC-catalyzed hydrolysis of the present invention to a patient in need of therapy that includes a bacterial infection with a Carbapenem-resistant bacterial that produces a KPC carbapenemase. The carbapenem antibiotic that is resistant to KPC-catalyzed hydrolysis may also be administered as any one of known salt forms.

The carbapenem antibiotic that is resistant to KPC-catalyzed hydrolysis is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the carbapenem antibiotic that is resistant to KPC-catalyzed hydrolysis may be formulated to provide, e.g., maximum and/or consistent dosing for the particular form for oral, rectal, topical, intravenous injection or parenteral administration. While the carbapenem antibiotic that is resistant to KPC-catalyzed hydrolysis may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopocia, Thirty-Second Edition (The Pharmaceutical Press, London, 1999); all of which are incorporated by reference, and the like, relevant portions incorporated herein by reference.

For example, the carbapenem antibiotic that is resistant to KPC-catalyzed hydrolysis may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The carbapenem antibiotic that is resistant to KPC-catalyzed hydrolysis may be administered in the form of liposome delivery systems, e.g., small unilamellar vesicles, large unilamellar vesicles, and multilamellar vesicles, whether charged or uncharged. Liposomes may include one or more: phospholipids (e.g., cholesterol), stearylamine and/or phosphatidylcholines, mixtures thereof, and the like.

That carbapenem antibiotic that is resistant to KPC-catalyzed hydrolysis may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the carbapenem antibiotic that is resistant to KPC-catalyzed hydrolysis may be coupled one or more biodegradable polymers to achieve controlled release of the carbapenem antibiotic that is resistant to KPC-catalyzed hydrolysis, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the carbapenem antibiotic that is resistant to KPC-catalyzed hydrolysis and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl-or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagus, tachea, lungs and alveoli, the carbapenem antibiotic that is resistant to KPC-catalyzed hydrolysis may also be delivered as an intranasal form via use of a suitable intranasal vehicle. For dermal and transdermal delivery, the carbapenem antibiotic that is resistant to KPC-catalyzed hydrolysis may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of carbapenem antibiotic that is resistant to KPC-catalyzed hydrolysis may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of carbapenem antibiotic that is resistant to KPC-catalyzed hydrolysis. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

As used herein, the term “chewable” refers to semi-soft, palatable and stable chewable treat without addition of water. It should be appreciated to the skilled artisan that a chewable composition will be stable and palatable, fast disintegrating, semi-soft medicated chewable tablets (treats) by extrusion without the addition of extraneous water. A soft chewable tablet does not harden on storage and are resistant to microbial contamination. A semi-soft chewable contains a blend of any one or more of binders, flavours, palatability enhancers, humectants, disintegrating agents, non-aqueous solvents, and diluents that are plasticized with liquid plasticizers, such as glycols and polyols to make them ductile and extrudable. The chewable can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorings and/or taste-masking agents that improve the taste of the formulation by greater than 10, 20, 30, 40, 50, 60, 70, 80, or 90%. The chewable can include the active agent and the ion exchange resin to enhance taste masking.

For topical administration, the composition can be incorporated into creams, ointments, gels, transdermal patches, and the like. The composition can also be incorporated into medical dressings, for example, wound dressings, e.g., woven (e.g., fabric) dressings, or non-woven dressings (e.g., gels or dressings with a gel component). The use of alginate polymers in dressings is known, and such dressings, or indeed any dressings, may further incorporate the alginate oligomers of the invention.

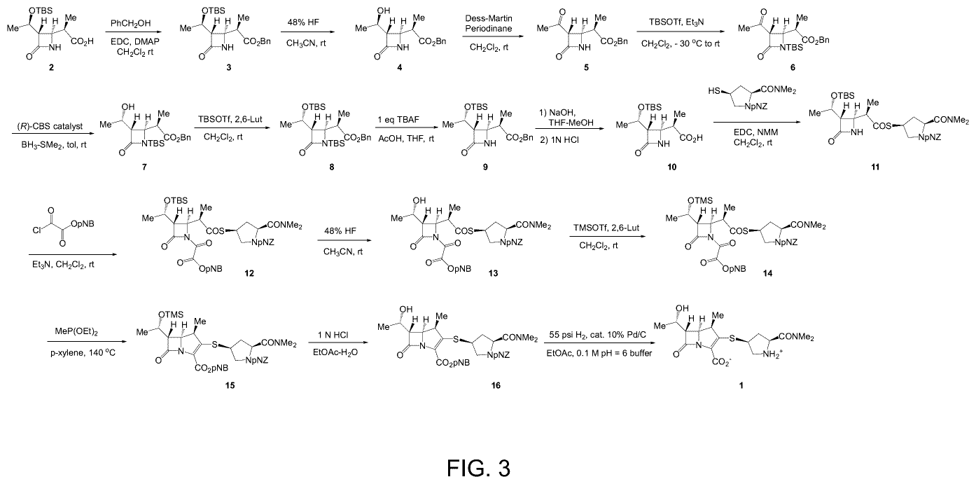

The synthetic methodology which has been employed to make these new carbapenems is shown in FIG. 3 (Example 1 of general structure).

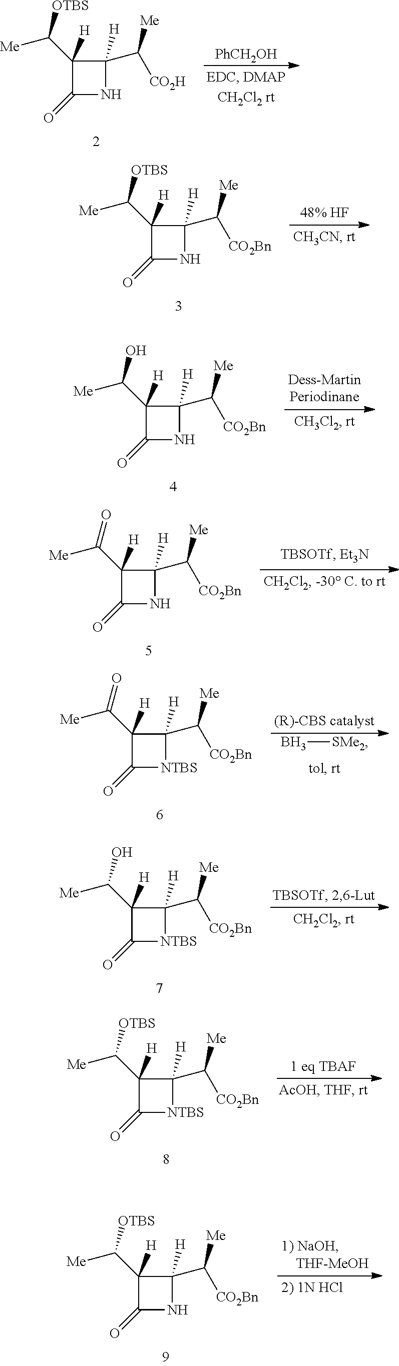

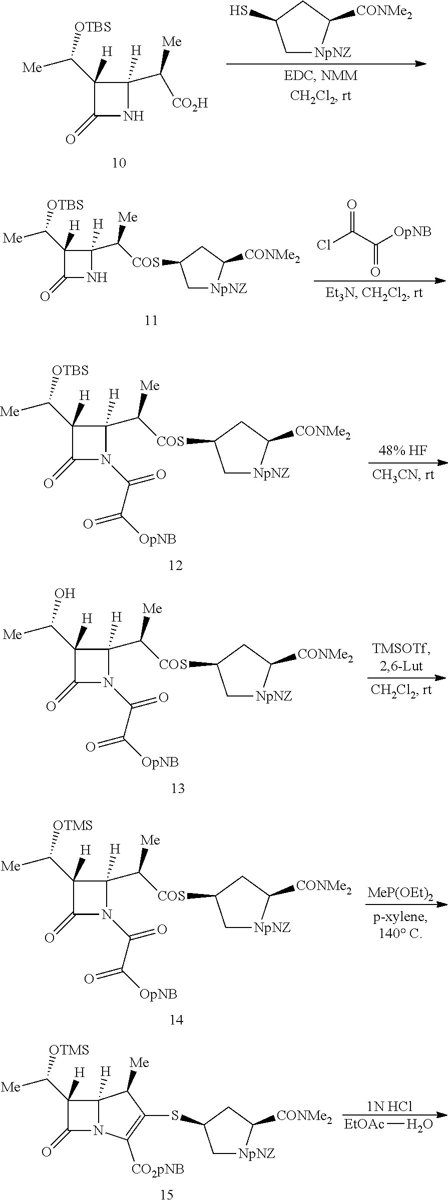

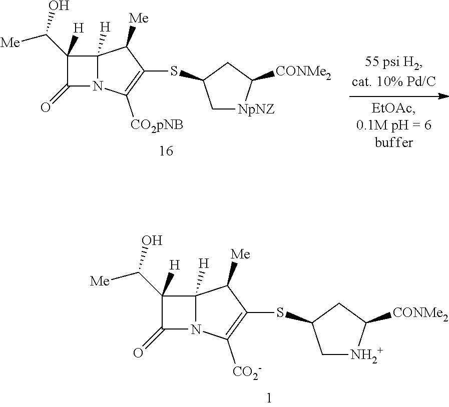

### Experimental Section, FIG. 3

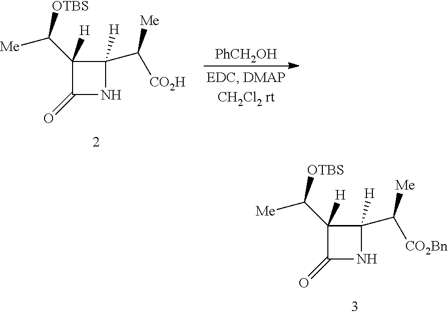 [SMILES: "PhCH2OH" ; C[C@@H](O[Si](C)(C)C(C)(C)C)[C@H]1C(=O)N[C@@H]1[C@@H](C)C(=O)O ; C[C@@H](O[Si](C)(C)C(C)(C)C)[C@H]1C(=O)N[C@@H]1[C@@H](C)C(=O)OCc1ccccc1]

### (2R)-2-[(2S,3S)-3-[(1R)-1-[(tert-butyldimethylsilyl)oxy]ethyl]-4-oxoazetidin-2-yl]propanoate

Commercial (2) (20 g, 66.3 mmol) was dissolved in 400 mL dry DCM. Benzyl alcohol (7.17 g, 66.3 mmol) and DMAP (12.15 g, 99.5 mmol) were added to the reaction at 0° C. and stirred for 1 hour. EDC (25.4 g, 133 mmol) was then added very slowly and the reaction allowed to warm to room temperature. After the reaction was completed (3 hours), the organic layer washed with 1N HCl, water, brine, then separated and concentrated. The residue was purified by flash chromatography (DCM/EtOAc) to afford 22 g (85%) compound 3. ¹H NMR (400 MHz, CDCl₃): δ 6.322 (s, 1H), 4.182 (t, H), 3.911 (d, H), 2.982 (s, 1H), 2.747 (t, 1H) ¹³C NMR (400 MHz, CDCl₃): δ 173.69, 168.90, 135.63, 128.54, 128.26, 128.10, 66.46, 65.10, 61.88, 52.05, 43.25, 25.94, 25.79, 22.37, 18.24, 17.91, 17.57, 14.17, 13.10, −4.07, −4.35, −4.62, −4.69, −4.97, −5.25 IR (KBr, cm⁻¹): 3448.05, 2962.41, 2850.79, 2284.30, 1762.84, 1734.46, 1499.26, 1458.10, 1378.53, 1336.84, 1258.27, 1162.64, 1100.77, 1043.49, 959.22, 800.73, 691.68.

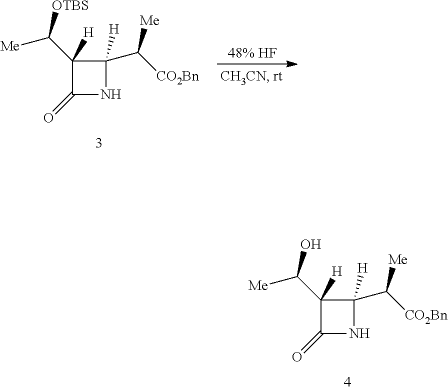 [SMILES: C[C@@H](O[Si](C)(C)C(C)(C)C)[C@H]1C(=O)N[C@@H]1[C@@H](C)C(=O)OCc1ccccc1 ; C[C@@H](O)[C@H]1C(=O)N[C@@H]1[C@@H](C)C(=O)OCc1ccccc1]

Benzyl (2R)-2-[(2S,3S)-3-[(1R)-1-hydroxyethyl]-4-oxoazetidin-2-yl]propanoate 48% HF (4 mL, 110 mmol) was added to a solution of compound 3 (22 g, 56.2 mmol) in 50 mL CH₃CN at room temperature. The reaction was monitored by thin layer chromatography (TLC). Additional 4 mL 48% HF was added to the reaction to complete. After the reaction was completed, fine powder of NaHCO3 was added to the mixture to adjust pH to 7. The solid was filtered and the filtrate was concentrated under reduced pressure to obtain 14.3 g (92%) white solid 4. ¹H NMR (400 MHz, CDCl₃): δ 7.346 (m, 5H), 6.30 (s, 1H), 4.130 (m, 3H), 3.761 (d, 3H), 3.440 (s, 1H), 2.953 (d, 3H), 2.627 (t, 1H), 1.276 (d, 3H). ¹³C NMR (400 MHz, CDCl₃): δ 174.26, 167.93, 135.22, 128.67, 128.54, 128.31, 66.98, 65.68, 62.46, 53.59, 43.28, 21.13, 13.71.

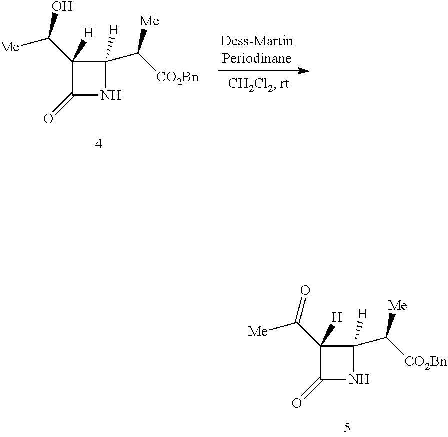 [SMILES: C[C@@H](O)[C@H]1C(=O)N[C@@H]1[C@@H](C)C(=O)OCc1ccccc1 ; CC(=O)[C@H]1C(=O)N[C@@H]1[C@@H](C)C(=O)OCc1ccccc1]

### Benzyl (2R)-2-[(2S,3S)-3-acetyl-4-oxoazetidin-2-yl]propanoate

Alcohol 4 (14.3 g, 51.6 mmol) was dissolved in 250 mL dry DCM at room temperature. DMP (26.7 g, 63 mmol) was added slowly over 30 minutes. The reaction stirred for 15 minutes at room temperature. A solution of Na₂S₂O₃·5H₂O in a saturated solution of NaHCO₃ was added to the reaction and stirred for an additional hour till DMP disappeared as monitored by the ¹H NMR spectra. The organic layer was separated and washed with water. Then, the organic layer was concentrated under reduced pressure to obtain 13 g (92%) compound 5 which was used immediately, without further purification, in the next reaction. ¹H NMR (400 MHz, CDCl₃): δ 7.346 (m, 5H), 6.238 (s, 1H), 5.163 (s, 2H), 4.212 (dd, 1H), 4.113 (d, 1H), 2.770 (m, 1H), 2.211 (s, 3H), 1.279 (d, 3H). ¹³C NMR (400 MHz, CDCl₃): δ 199.24, 172.83, 163.05, 135.17, 128.50, 66.88, 42.04, 29.61, 12.75.

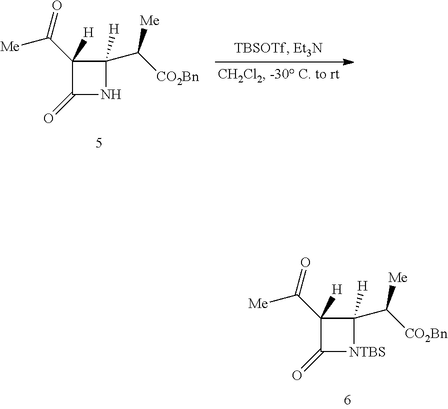

### Benzyl (R)-2-((2S,3S)-3-acetyl-1-(tert-butyldimethylsilyl)-4-oxoazetidin-2-yl)propanoate

A −30° C. solution of the ketone 5 (6.0 g, 21.8 mmol) in 100 mL dry DCM was treated with triethylamine (6.07 mL, 4.41 g, 43.6 mmol) and then with tert-butyldimethylsilyl trifluoromethanesulfonate (TBS-OTf) (6.33 g, 5.51 mL, 24 mmol). The reaction mixture was stirred at 0° C. and gradually to room temperature under nitrogen for 30 minutes and was then washed with saturated aq NaHCO₃, H₂O, brine, dried over Na₂SO₄, and evaporated to dryness. The residue was purified by a flash chromatography (DCM) to give 7.5 g (88%) the title compound (6). ¹H NMR (400 MHz, CDCl₃): δ 7.336 (m, 5H), 5.348 (s, 2H), 4.212 (dd, 1H), 3.813 (d, 1H), 2.950 (m, 1H), 2.311 (s, 3H), 1.129 (d, 3H). ¹³C NMR (400 MHz, CDCl₃): δ 203.11, 172.83, 168.45, 136.17, 128.90, 66.78, 58.72, 48.61, 43.34, 30.91, 27.02, 11.75.

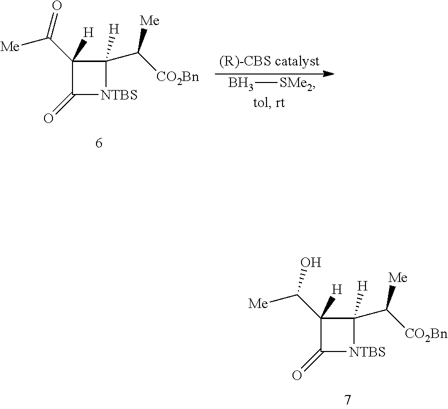

### Benzyl (R)-2-((2S,3S)-1-(tert-butyldimethylsilyl)-3-((S)-1-hydroxyethyl)-4-oxoazetidin-2yl)propanoate

To a solution of (R)-(+)-2-methyl-CBS-oxazaborolidine (8.87 g, 32 mmol) in 80 mL dry toluene was added BH₃—SMe₂ (3.2 mL, 10 M, 32 mmol) at room temperature. This solution was allowed to stir at room temperature for 30 minutes, then cooled to 0° C. and treated with a solution of ketone 6 (12.5 g, 32 mmol) in 100 mL dry toluene. After stirring for 1 hour at 0° C., the reaction mixture was treated with MeOH (caution, vigorous evolution of gas!) to quench remaining borane. The solution was poured into water and extracted with EtOAc. Organic layers were dried over Na₂SO₄, concentrated and the residue was purified by silica gel chromatography to give 7.2 g (57%) the titled compound 7. ¹H NMR (400 MHz, CDCl₃): δ 7.336 (m, 5H), 6.771 (s, 1H), 5.348 (s, 2H), 3.902 (m, 1H), 3.900 (m, 1H), 2.950 (m, 1H), 1.120 (d, 3H), 0.989 (d, 3H). ¹³C NMR (400 MHz, CDCl₃): δ 173.01, 172.23, 136.17, 128.90, 67.78, 66.71, 53.72, 48.81, 44.34, 30.91, 27.02, 20.61, 11.75.

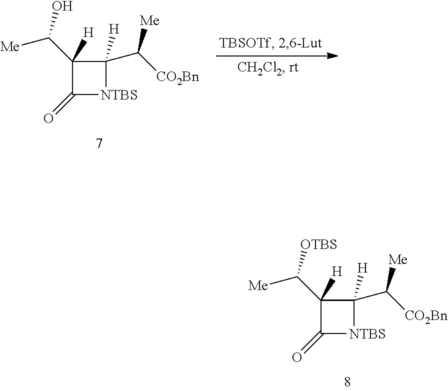

### Benzyl (R)-2-((2S,3S)-1-(tert-butyldimethylsilyl)-3-((S)-1-((tert-butyldimethylsilyl)oxy)ethyl)-4-oxoazetidin-2-yl)propanoate

A 0° C. solution of 7 (3.7 g, 9.45 mmol) in 50 mL dry DCM was treated with 2,6-lutidine (2.54 g, 2.75 mL, 23.7 mmol) and then with tert-butyldimethylsilyl trifluoromethanesulfonate (4.01 g, 3.49 mL, 15.2 mmol). The reaction mixture was stirred at 0° C. under N₂ for 30 min and was then washed with saturated NaHCO₃, H₂O, brine, dried over Na₂SO₄, and evaporated to dryness. The residue was purified by flash chromatography (DCM) to give 4.1 g (86%) of the title compound 8. ¹H NMR (400 MHz, CDCl₃): δ 7.336 (m, 5H), 5.348 (s, 2H), 3.902 (m, 1H), 3.900 (m, 1H), 2.950 (m, 1H), 1.220 (dd, 3H), 0.989 (d, 3H), 0.212 (d, 3H), 0.081 (d, 3H). ¹³C NMR (400 MHz, CDCl₃): δ 173.01, 172.23, 136.17, 128.90, 69.00, 66.71, 54.02, 48.81, 44.34, 30.91, 27.02, 22.91, 11.75.

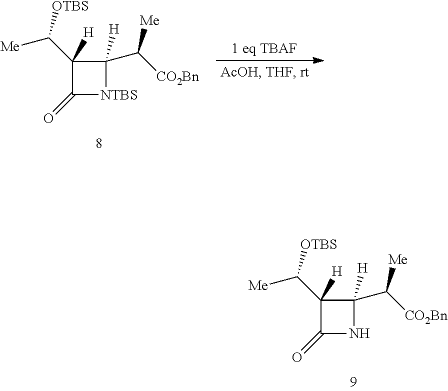

### Benzyl (R)-2-((2S,3S)-3-((S)-1-((tert-butyldimethylsilyl)oxy)ethyl)-4-oxoazetidin-2-yl)propanoate

Compound 8 (2.6 g, 5.14 mmol) was dissolved in 28 mL THF. Acetic acid (1.54 g, 1.47 mL, 25.7 mmol) and tetra butylammonium fluoride (TBAF) (5.14 mL, 1M THF, 5.14 mmol) were added at 0° C. under nitrogen (N₂) atmosphere for 30 minutes. The reaction was monitored by TLC and stirred for 3 hours. After the reaction completed, EtOAc was added to the reaction and the organic layer was washed with water, brine, dried over Na₂SO₄ and concentrated to give 1.9 g (94%) the title compound 9. ¹H NMR (400 MHz, CDCl₃): δ 7.336 (m, 5H), 5.348 (s, 2H), 3.902 (m, 1H), 3.850 (m, 1H), 2.950 (m, 1H), 1.120 (dd, 3H), 1.220 (dd, 3H), 0.989 (m, 3H), 0.212 (d, 3H). ¹³C NMR (400 MHz, CDCl₃): δ 173.01, 169.93, 136.17, 128.90, 69.00, 66.71, 53.02, 46.81, 43.34, 30.91, 25.92, 22.91, 11.75.

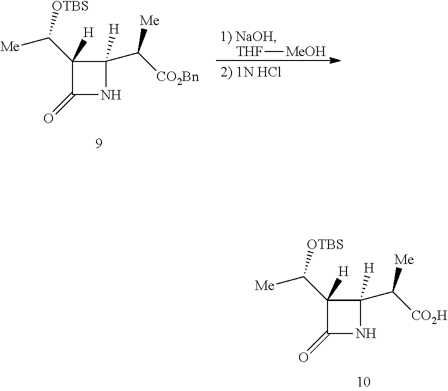

### (R)-2-((2S,3S)-3-((S)-1-((tert-butyldimethylsilyl)oxy)ethyl)-4-oxoazetidin-2-yl)propanoic acid

Compound 9 (1.6 g, 4.09 mmol) was dissolved in 18 mL THF. IN sodium hydroxide (4.23 mL, 4.23 mmol) and methanol (4.23 mL) were added to the reaction mixture and heated between 40° C. and 50° C. for 1 hour. After the reaction was completed, solvents were removed under reduced pressure. Water was added to the residue and the aqueous solution was washed with ether to remove the benzyl alcohol. IN HCl and ethyl acetate was added to the aqueous layer and stirred till pH=2. The organic layer was dried over Na₂SO₄, and evaporated to dryness to give 1.0 g (81%) of the title compound 10. ¹H NMR (400 MHz, CDCl₃): δ 10.631 (s, 1H), 3.902 (m, 1H), 3.540 (m, 1H), 2.998 (m, 1H), 2.880 (m, 1H), 1.220 (dd, 3H), 1.070 (dd, 3H), 0.989 (m, 3H), 0.212 (d, 3H). ¹³C NMR (400 MHz, CDCl3): δ 178.31, 169.93, 69.00, 53.01, 45.77, 45.31, 30.92, 25.97, 22.91, 11.50.

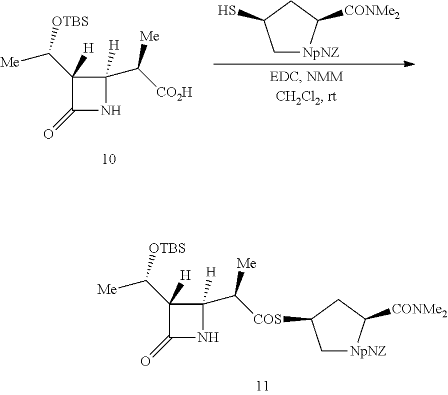

### (2S,4S)-4-(((R)-2-((2S,3S)-3-((S)-1-((tert-butyldimethylsilyl)oxy)ethyl)-4-oxoazetidin-2 yl)propanoyl)thio)-2-(dimethylcarbamoyl)pyrrolidine-1-carboxylic 4-nitrobenzoic anhydride

Compound 10 (1.0 g, 3.32 mmol) dissolved in 20 mL dry DCM. Thiol (1.41 g, 3.99 mmol), N-methylmorpholine (0.672 g, 0.732 mL, 3.99 mmol), 4-dimethylaminopyridine (0.406 g, 3.32 mmol) and 1-ethyl-3-(3-dimethylaminopropyl) carbodiimide hydrochloride (1.27 g, 6.63 mmol) were added to the reaction at 0° C. for an hour. After the reaction completed, the organic layer washed with 1N HCl, water, brine, then separated and concentrated. The residue was purified by flash chromatography (DCM/MeOH) to afford 2.0 g (95%) of compound 11. ¹H NMR (400 MHz, CDCl₃): δ 8.481 (m, 1H), 4.392 (m, 1H), 4.210 (m, 2H), 3.908 (m, 1H), 2.980 (q, 3H), 2.790 (dd, 2H), 1.220 (dd, 3H), 1.120 (dd, 3H), 0.989 (m, 3H), 0.212 (d, 3H). ¹³C NMR (400 MHz, CDCl₃): δ 204.24, 169.91, 159.39, 153.11, 134.35, 131.21, 123.81, 63.52, 53.13, 50.21, 47.21, 38.23, 33.80, 30.91, 27.99, 25.90, 22.93, 13.02.

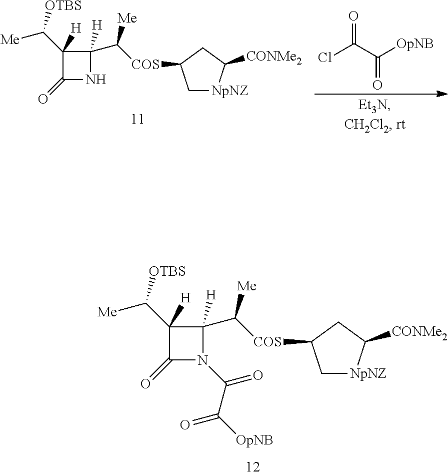

(2R,4R)-4-(((S)-2-((2S,3S)-3-((S)-1-((tert-butyldimethylsilyl)oxy)ethyl)-1-(2-((4-nitrobenzoyl)oxy)-2-oxoacetyl)-4-oxoazetidin-2-yl)propanoyl)thio)-2-(dimethylcarbamoyl)pyrrolidine-1-carboxylic 4-nitrobenzoic anhydride

At 0° C., to a solution of compound 11 (2.0 g, 3.14 mmol) in 30 mL dry DCM, triethylamine (0.479 g, 0.659 mL, 4.73 mmol), and p-nitrobenzyl oxalyl chloride (0.989 g, 4.06 mmol) were added to the reaction. After the reaction completed, the organic layer was washed with H₂O, aq NaHCO₃, dried over Na₂SO₄ and concentrated under reduced pressure. The residue was purified by flash chromatography (DCM) to give 2.0 g (75%) of the title compound (12). ¹H NMR (400 MHz, CDCl₃): δ 8.481 (m, 1H), 4.392 (m, 1H), 4.210 (m, 2H), 3.908 (m, 1H), 3.101 (s, 1H), 2.980 (d, 3H), 2.790 (dd, 2H), 1.220 (dd, 3H), 1.120 (dd, 3H), 0.989 (m, 3H), 0.212 (d, 3H). ¹³C NMR (400 MHz, CDCl₃): δ 204.24, 169.91, 159.39, 155.31, 153.11, 134.35, 131.21, 123.81, 69.01, 63.52, 53.13, 50.21, 47.21, 38.23, 33.80, 30.91, 27.99, 25.90, 22.93, 13.02.

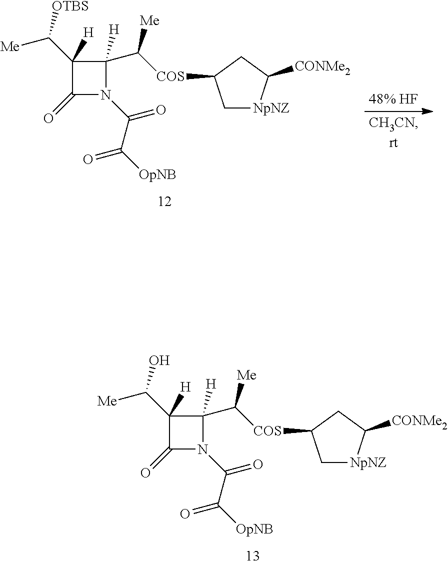

### (2R,4R)-2-(dimethylcarbamoyl)-4-(((S)-2-((2S,3S)-3-((S)-1-hydroxyethyl)-1-(2-((4-nitrobenzoyl)oxy)-2-oxoacetyl)-4-oxoazetidin-2-yl)propanoyl)thio)pyrrolidine-1-carboxylic 4-nitrobenzoic anhydride

The beta lactam 12 (2.0 g, 2.37 mmol) was dissolved in 30 mL CH₃CN. 48% HF (8 mL, 220 mmol) was added to the reaction and stirred at room temperature. When the reaction was complete as monitoring by tlc, aq. NaHCO₃ was added to the reaction until it reached pH=8 and concentrated to give 1.6 g (92%) of the titled compound 13. ¹H NMR (400 MHz, CDCl₃): δ 8.481 (m, 1H), 6.777 (dd, 1H), 4.392 (m, 1H), 4.210 (m, 2H), 3.908 (m, 1H), 3.101 (s, 1H), 2.980 (d, 3H), 2.790 (dd, 2H), 1.120 (dd, 3H), 1.120 (dd, 3H). ¹³C NMR (400 MHz, CDCl3): δ 204.24, 169.91, 159.39, 155.31, 153.11, 134.35, 131.21, 123.81, 67.61, 63.52, 51.13, 50.21, 49.21, 38.23, 33.80, 27.99, 20.93, 13.02.

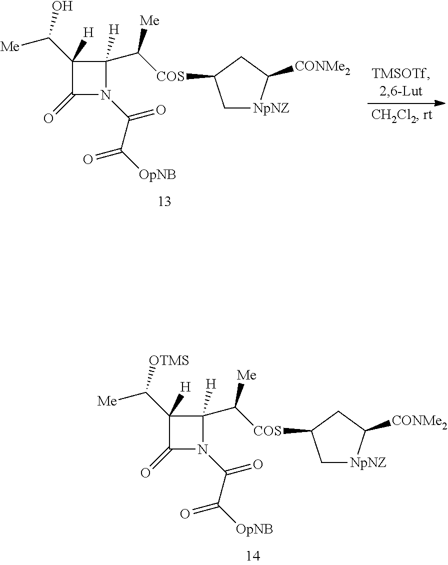

(2R,4R)-2-(dimethylcarbamoyl)-4-(((S)-2-((2S,3S)-1-(2-((4-nitrobenzoyl)oxy)-2-oxoacetyl)-4-oxo-3-((S)-1-((trimethylsilyl) oxy) ethyl) azetidin-2-yl)propanoyl)thio)pyrrolidine-1-carboxylic 4-nitrobenzoic anhydride

At 0° C., 2,6-lutidine (0.47 g, 0.508 mL, 4.39 mmol) and TMS-OTf (0.731 g, 0.599 mL, 3.29 mmol) were added to a solution of the beta lactam 13 (1.6 g, 2.19 mmol) in 25 mL dry DCM. The reaction was stirred at this temperature until complete by tlc. The reaction was washed with NaHCO₃, dried over Na₂SO₄ and evaporated under reduced pressure to give 1.28 g (73%) of the titled compound 14. ¹H NMR (400 MHz, CDCl₃): δ 8.481 (m, 1H), 4.392 (m, 1H), 4.210 (m, 2H), 3.908 (m, 1H), 3.101 (s, 1H), 2.980 (d, 3H), 2.790 (dd, 2H), 1.220 (dd, 3H), 1.120 (dd, 3H), 0.212 (d, 3H). ¹³C NMR (400 MHz, CDCl₃): δ 204.21, 169.91, 159.39, 155.31, 153.11, 134.35, 131.21, 123.81, 68.16, 63.52, 51.13, 50.21, 48.21, 38.23, 33.80, 27.99, 22.93, 13.02, 3.91

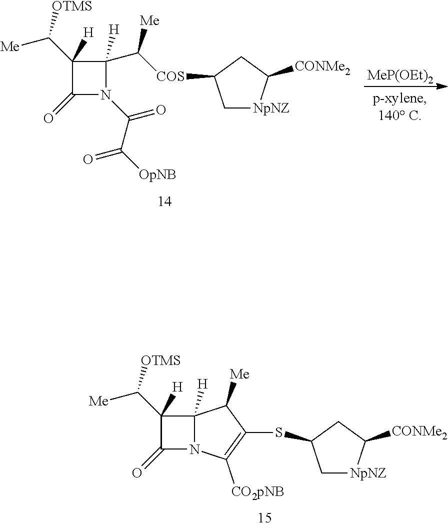

(4R,5S,6S)-3-(((3S,5S)-1-(((4-((λ1-oxidaneyl)diazenyl)benzyl)oxy)carbonyl)-5-(dimethylcarbamoyl)pyrrolidin-3-yl)thio)-4-methyl-7-oxo-6-((S)-1-((trimethylsilyl)oxy)ethyl)-1-azabicyclo[3.2.0]hept-2-ene-2-carboxylic 4-nitrobenzoic anhydride

MeP(OEt)₂ (0.726 g, 0.8 mL, 5.33 mmol) was added to a dry flask that contained a toluene (5mL) solution of compound 14 (1.28 g, 1.60 mmol). The mixture was stirred under N₂ at room temperature for 1 hour. The completion of the reaction was monitored by TLC. The toluene and excess MeP(OEt)₂ was removed under high vacuum. Then, 50 mL dry p-xylene was added and heated to reflux (140° C.) for 2 hours. The reaction mixture was concentrated to dryness. The crude product was purified by flash chromatography to give 0.8 g (65%) of the titled compound 15. ¹H NMR (400 MHz, CDCl₃): δ 8.481 (m, 1H), 7.871 (d, 1H), 7.322 (d, 1H), 5.021 (s, 2H), 4.392 (m, 1H), 4.210 (m, 2H), 3.908 (m, 1H), 3.101 (s, 1H), 2.980 (d, 3H), 2.790 (s, 1H), 2.790 (dd, 2H), 1.220 (dd, 3H), 1.090 (dd, 3H), 0.212 (d, 3H). ¹³C NMR (400 MHz, CDCl₃): δ 165.91, 153.11, 143.35, 142.21, 134.31, 129.00, 123.82 109.81, 67.16, 63.52, 53.13, 44.21, 42.23, 38.21, 32.80, 30.79, 3.91

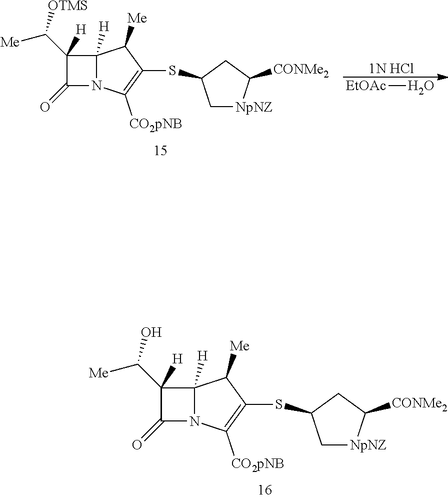

(4R,5S,6S)-3-(((3S,5S)-1-(((4-((λ1-oxidaneyl)diazenyl)benzyl)oxy)carbonyl)-5-(dimethylcarbamoyl)pyrrolidin-3-yl)thio)-6-((S)-1-hydroxyethyl)-4-methyl-7-oxo-1-azabicyclo[3.2.0]hept-2-ene-2-carboxylic 4-nitrobenzoic anhydride

A solution of 15 (0.8 g, 1.04 mmol) in 30 mL EtOAc was treated with 10 mL IN HCl. After the reaction completed, the layers were separated, the solvent was removed in vacuo. The residue was diluted with EtOAc and washed with H₂O and NaHCO₃. The organic layer was dried over anhydrous Na₂SO₄ and evaporated under reduced pressure. The product was clean without further purification to give 0.55 g (76%) titled compound 16. ¹H NMR (400 MHz, CDCl₃): 8 8.481 (m, 1H), 7.871 (d, 1H), 7.322 (d, 1H), 6.772 (s, 1H), 5.021 (s, 2H), 4.392 (m, 1H), 4.210 (m, 2H), 3.908 (m, 1H), 3.101 (s, 1H), 2.980 (d, 3H), 2.800 (s, 1H), 2.790 (dd, 2H), 1.110 (dd, 3H), 1.090 (dd, 3H). ¹³C NMR (400 MHz, CDCl₃): δ 169.91, 165.92, 160.02, 152.11, 143.35, 142.21, 134.31, 129.00, 123.82 109.81, 68.02, 67.16, 63.52, 53.13, 42.23, 38.21, 32.80, 30.79, 20.62, 13.11

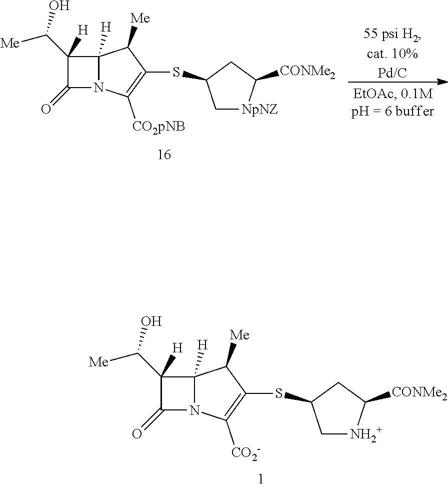

### (4R,5S,6S)-3-(((3S,5S)-5-(dimethylcarbamoyl)pyrrolidin-1-ium-3-yl)thio)-6-((S)-1-hydroxyethyl)-4-methyl-7-oxo-1-azabicyclo[3.2.0]hept-2-ene-2-carboxylate

A solution of 16 (0.55 g, 0.788 mmol) in EtOAc (40 mL) and 1M sodium phosphate buffer pH ˜6.0 (40 mL) was treated 10% Pd/C (0.7 g). The mixture was treated with H₂ gas in a Parr hydrogenation vessel at 55 psi for 1.5 hours. The mixture was filtered through Celite pad and washed with small amounts of EtOAc and HPLC H₂O. The aqueous layer was collected, washed with ether, and concentrated under reduced pressure. The crude mixture was purified by flash chromatography (diaion resin, 0 to 40% EtOH in H₂O) to yield 0.16 g (53%) the titled antibiotic (1, JDB-1-200). ¹H NMR (400 MHz, CDCl₃): δ 6.772 (s, 1H), 4.422 (m, 1H), 4.061 (s, 2H), 3.908 (m, 1H), 3.061 (s, 1H), 2.980 (d, 3H), 2.800 (s, 1H), 2.755 (d, 2H) 1.110 (dd, 3H), 1.090 (dd, 3H). ¹³C NMR (400 MHz, CDCl₃): δ 177.21, 173.92, 165.92, 148.31, 122.22, 68.02, 63.52, 55.53, 52.23, 44.21, 42.50, 40.69, 38.22, 20.11, 13.22

### Results and Discussion

TABLE 1
In vitro activity of SMU test articles
against Gram-negative aerobic bacteria.

Organism

| Name | MIC (μg/mL) | | | | | | |
|---|---|---|---|---|---|---|---|
| Source No. | JDB-1- | | | | | | |
| and Type | 200 (1) | MEM | CAZ | MOX | VAN | COL | MET |
| Klebsiella | 1 | >8 | >32 | >4 | >32 | 0.12 | >32 |
| pneumoniae | | | | | | | |
| MMX 4683 | | | | | | | |
| (CDC | | | | | | | |
| ART2008030); | | | | | | | |
| ESBL, KPC-2 | | | | | | | |
| Klebsiella | 0.5 | >8 | >32 | >4 | >32 | 8 | >32 |
| pneumoniae | | | | | | | |
| MMX 4885; | | | | | | | |
| KPC | | | | | | | |
| Klebsiella | 0.5 | 0.25 | 0.5 | 0.5 | >32 | 0.25 | >32 |
| pneumoniae | | | | | | | |
| MMX | | | | | | | |
| 6854; ESBL | | | | | | | |
| Enterobacter | 0.12 | 0.06 | 32 | 0.03 | >32 | 0.12 | >32 |
| cloacae | | | | | | | |
| MMX 6089 | | | | | | | |
| Citrobacter | 1 | >8 | >32 | >4 | >32 | 0.12 | >32 |
| freundii | | | | | | | |
| MMX 6284 | | | | | | | |
| Proteus | 1 | 0.12 | ≤0.03 | 0.25 | >32 | >32 | >32 |
| mirabilis | | | | | | | |
| MMX 6442 | | | | | | | |
| Serratia | 0.5 | 0.25 | 0.25 | 0.12 | >32 | >32 | >32 |
| marcescens | | | | | | | |
| MMX 6462 | | | | | | | |
| Acinetobacter | 0.5 | 2 | 8 | 0.25 | >32 | 0.5 | >32 |
| baumannii | | | | | | | |
| ATCC 19606 | | | | | | | |
| Acinetobacter | 32 | >8 | >32 | >4 | >32 | 1 | >32 |
| baumannii | | | | | | | |
| MMX | | | | | | | |
| 2587; MDR | | | | | | | |
| Acinetobacter | 0.12 | 0.25 | 8 | 0.12 | >32 | 2 | >32 |
| baumannii | | | | | | | |
| MMX 2599 | | | | | | | |

ESBL, extended-spectrum β-lactamase positive; KPC, Klebsiella pneumoniae carbapenemase; COL-R; colistin-resistant; MDR, multidrug-resistant. MEM, meropenem; CAZ, ceftazidime; MOX, moxifloxacin; VAN, vancomycin; COL, colistin; MET, metronidazole.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertic(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### REFERENCES

Íñigo, M.; Del Pozo, J. L., Treatment of infections caused by carbapenemase-producing Enterobacterales. Rev Esp Quimioter 2022, 35 Suppl 3 (Suppl 3), 46-50.

What is claimed is:

1. A compound of Formula I, or a pharmaceutically acceptable salt or ester thereof: 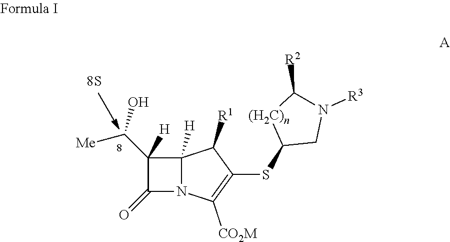
R¹=H or CH₃;
n=0 or 1;
R² is H, CH₂NHSO₂NH₂, or CONR^{a}R^{b}, where R^{a} and R^{b} may be independently equal to H, Me, Aryl, or substituted Aryl; and
R³ is H, dihydrothiazole, C1 to C6 Alkyl, or R³=COR^{c}, where R^{c}=C1 to 6 alkyl, aryl, substituted aryl, or R^{c}=NHR^{d}, where R^{d}=H, aryl or substituted aryl.

2. The compound of claim 1, wherein the compound is:

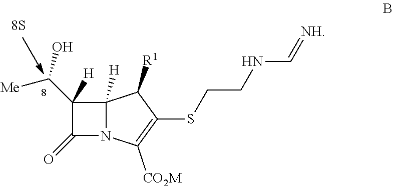

3. The compound of claim 1, wherein the —CO₂M, which is attached to the carbapenem nucleus at position 3, wherein M is a carboxylic acid group (M represents H), a carboxylate anion (M represents a negative charge), a pharmaceutically acceptable ester (M represents an ester forming group) or a carboxylic acid protected by a protecting group (M represents a carboxyl protecting group).

4. The compound of claim 1, wherein the pharmaceutically acceptable salts form —COOM, where M is a negative charge, which is balanced by a counterion, an alkali metal cation, sodium, potassium calcium, magnesium, zinc, ammonium, or alkylammonium cations such as tetramethylammonium, tetrabutylammonium, choline, triethylhydroammonium, meglumine, or triethanolhydroammonium; or the pharmaceutically acceptable salts comprise acid addition salts, inorganic, or organic acids.

5. The compound of claim 1, wherein the pharmaceutically acceptable salts comprise acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, or undecanoate.

6. The compound of claim 1, wherein at least one of:
the pharmaceutically acceptable ester is a hydrolyzable ester;
the hydrolyzable esters is biologically hydrolyzable, is suitable for oral administration, has good absorption through the stomach or intestinal mucosa, or resistance to gastric acid degradation; or
the hydrolyzable ester M is selected from at least one of: an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl.

7. The compound of claim 1, wherein the compound is selected from at least one of:

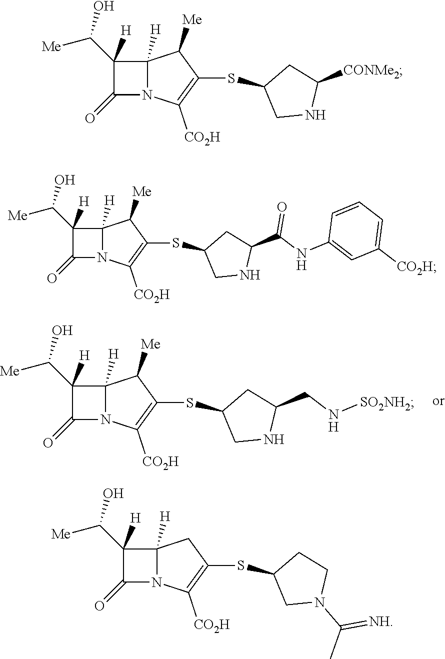

8. A method of making the compound of Formula I, comprising:

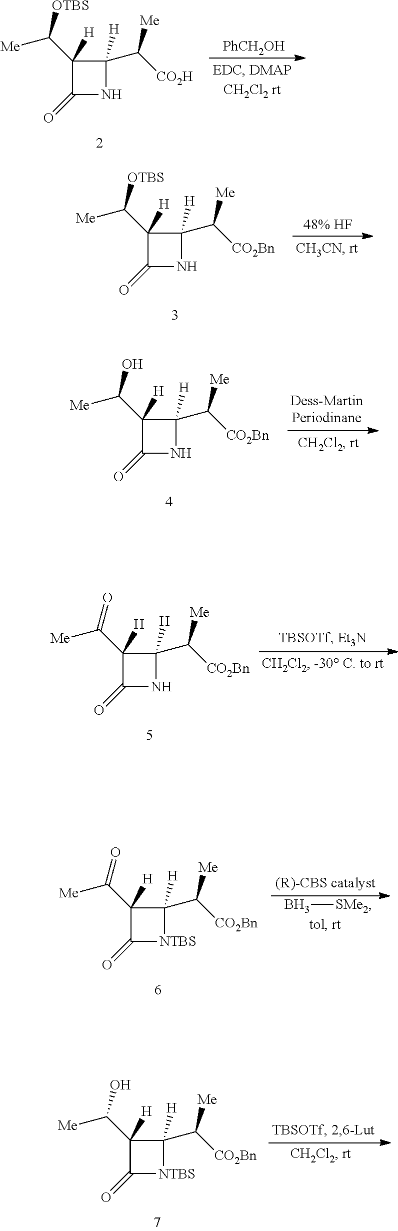

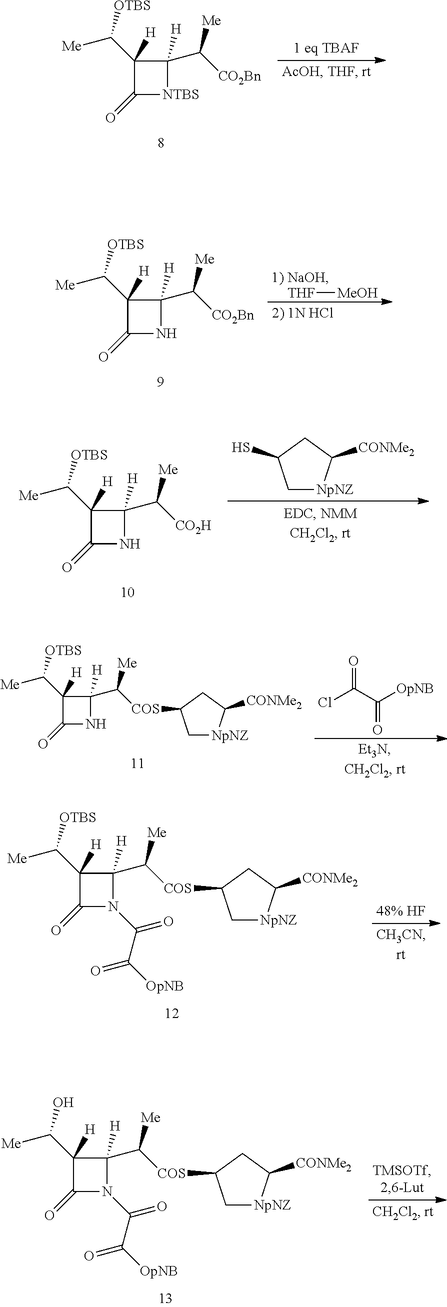

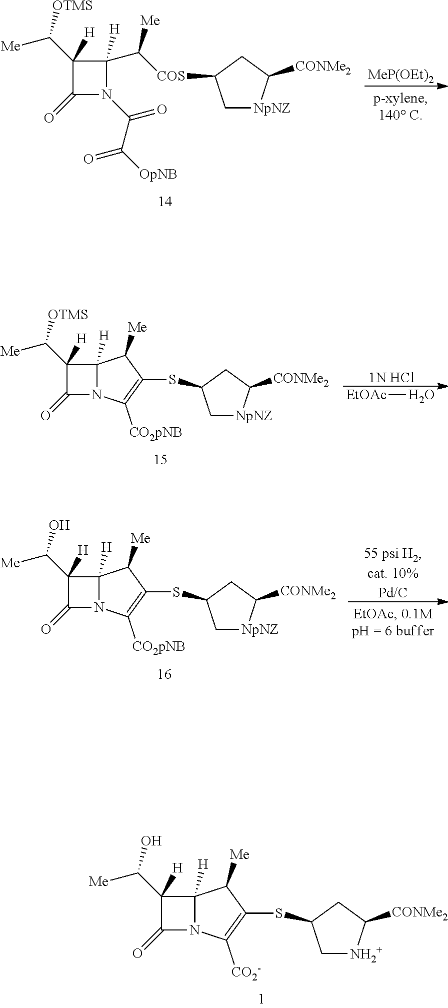

9. A composition comprising:
a compound of Formula I, or a pharmaceutically acceptable salt or ester thereof: 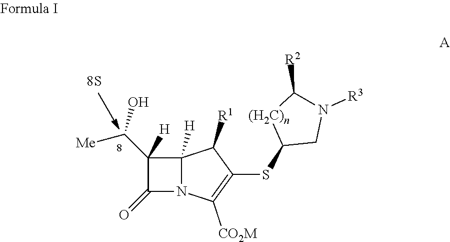
R¹=H or CH₃;
n=0 or 1;
R² is H, CH₂NHSO₂NH₂, or CONR^{a}R^{b}, where R^{a} and R^{b} may be independently equal to H, Me, Aryl, or substituted Aryl; and
R³ is H, dihydrothiazole, C1 to C6 Alkyl, or R³=COR^{c}, where R^{c}=C1 to 6 alkyl, aryl, substituted aryl, or R^{c}=NHR^{d}, where R^{d}=H, aryl or substituted aryl; and
a pharmaceutically acceptable carrier.

10. The composition of claim 9, wherein the compound is:

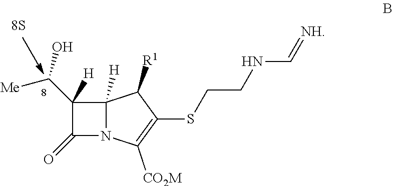

11. The composition of claim 9, wherein the —CO₂M, which is attached to the carbapenem nucleus at position 3, wherein M is a carboxylic acid group (M represents H), a carboxylate anion (M represents a negative charge), a pharmaceutically acceptable ester (M represents an ester forming group) or a carboxylic acid protected by a protecting group (M represents a carboxyl protecting group).

12. The composition of claim 9, wherein the pharmaceutically acceptable salts form-COOM, where M is a negative charge, which is balanced by a counterion, an alkali metal cation, sodium, potassium calcium, magnesium, zinc, ammonium, or alkylammonium cations such as tetramethylammonium, tetrabutylammonium, choline, triethylhydroammonium, meglumine, or triethanolhydroammonium; or the pharmaceutically acceptable salts comprise acid addition salts, inorganic, or organic acids.

13. The composition of claim 10, wherein the pharmaceutically acceptable salts comprise acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, or undecanoate.

14. The composition of claim 9, wherein at least one of:
the pharmaceutically acceptable ester is a hydrolyzable ester;
the hydrolyzable esters is biologically hydrolyzable, is suitable for oral administration, has good absorption through the stomach or intestinal mucosa, or resistance to gastric acid degradation; or
the hydrolyzable esters M is selected from at least one of: an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryI group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl.

15. The composition of claim 9, wherein the compound is at least one of:

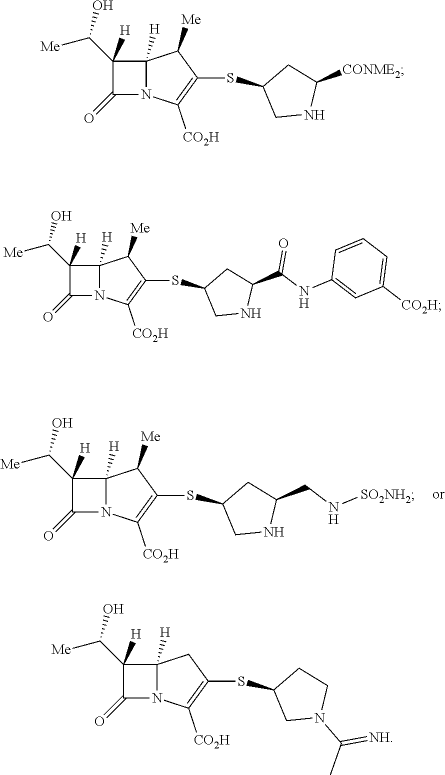

16. The composition of claim 9, further comprising an amount of a structurally modified carbapenem with carbapenemase inhibitors and synergistic mixtures of the compound of Formula I with other carbapenem antibiotics.

17. A method of treating a bacterial infection comprising:
contacting a bacteria with a compound of Formula I, or a pharmaceutically acceptable salt or ester thereof: 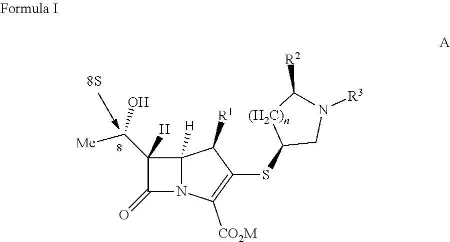
R¹=H or CH₃;
n=0 or 1;
R² is H, CH₂NHSO₂NH₂, or CONR^{a}R^{b}, where R^{a} and R^{b} may be independently equal to H, Me, Aryl, or substituted Aryl; and
R³ is H, dihydrothiazole, C1 to C6 Alkyl, or R³=COR^{c}, where R^{c}=C1 to 6 alkyl, aryl, substituted aryl, or R^{c}=NHR^{d}, where R^{d}=H, aryl or substituted aryl; and
a pharmaceutically acceptable carrier.

18. The method of claim 17, wherein the compound is:

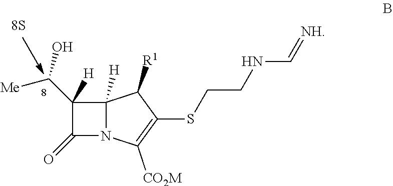

19. The method of claim 17, wherein the —CO₂M, which is attached to the carbapenem nucleus at position 3, wherein M is a carboxylic acid group (M represents H), a carboxylate anion (M represents a negative charge), a pharmaceutically acceptable ester (M represents an ester forming group) or a carboxylic acid protected by a protecting group (M represents a carboxyl protecting group).

20. The composition of claim 17, wherein the pharmaceutically acceptable salts form —COOM, where M is a negative charge, which is balanced by a counterion, an alkali metal cation, sodium, potassium calcium, magnesium, zinc, ammonium, or alkylammonium cations such as tetramethylammonium, tetrabutylammonium, choline, triethylhydroammonium, meglumine, or triethanolhydroammonium; or the pharmaceutically acceptable salts comprise acid addition salts, inorganic, or organic acids.

21. The method of claim 17, wherein the pharmaceutically acceptable salts comprise acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, or undecanoate.

22. The method of claim 17, wherein at least one of:
the pharmaceutically acceptable ester is a hydrolyzable ester;
the hydrolyzable esters is biologically hydrolyzable, is suitable for oral administration, has good absorption through the stomach or intestinal mucosa, or resistance to gastric acid degradation; or
the hydrolyzable esters M is selected from at least one of: an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl)methyl.

23. The method of claim 17, wherein the compound is at least one of:

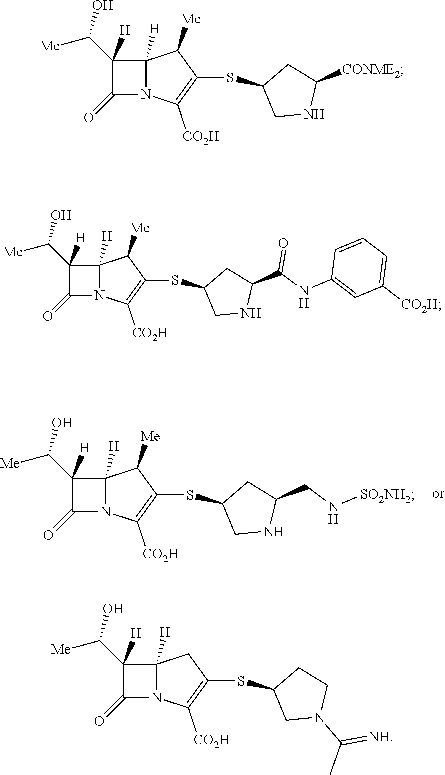

24. The method of claim 17, further comprising an amount of a structurally modified carbapenem with carbapenemase inhibitors and synergistic mixtures of the compound of Formula I with other carbapenem antibiotics.
